(19) United States
(12) Patent Application Publication
(10) Pub. No.: US 2026/0286336 A1
(43) Pub. Date: Sep. 24, 2026

(54) MAGNETIC HYDROGEL PARTICLE BASED MATRIX NORMALIZATION FOR INTACT MICROBIAL CAPTURE AND MOLECULAR DETECTION

(71) Applicant: CERES NANOSCIENCES, INC., MANASSAS, VA (US)

(72) Inventors: Anurag Patnaik, Manassas, VA (US); Ben Lepene, Manassas, VA (US); Patrick Andersen, Manassas, VA (US); Hien Le, Manassas, VA (US)

(21) Appl. No.: 19/571,007

(22) Filed: Mar. 18, 2026

Related U.S. Application Data
(60) Provisional application No. 63/773,806, filed on Mar. 18, 2025.

(51) Int. Cl. C12N 15/10; C12Q 1/6806

(52) U.S. Cl. CPC C12N 15/1003; C12Q 1/6806

(57) ABSTRACT

Described herein are methods, compositions, systems, and kits for capturing, concentrating, and analyzing microbial targets from diverse biological and environmental sample matrices. The disclosed methods employ magnetically responsive hydrogel particles in combination with a matrix conditioning composition to normalize physicochemical interactions within complex samples and enable capture of intact microbes across heterogeneous matrices. Following capture, the particles and associated microbial targets are magnetically separated to concentrate the microbes and reduce matrix-dependent variability prior to nucleic acid extraction, amplification, or detection. In certain embodiments, the methods facilitate processing of samples including, without limitation, whole blood, urine, wastewater, saliva, and respiratory transport media using a single standardized workflow. Captured microbial targets may be lysed on-particle or after separation from the sample matrix, and released nucleic acids may be purified for downstream molecular analysis.

This application claims priority to U.S. Provisional Patent Application number 63/773,806, filed on Mar. 18, 2025, titled “Magnetic hydrogel particle workflow to normalize matrix sample impact, enabling intact microbial capture resulting in improved detection, sensitivity, and reproducibility across analysis methods,” the contents of which are incorporated herein by reference in their entirety.

### FIELD OF THE PRESENT INVENTION

The present invention relates to microbial capture, concentration, extraction and molecular analysis. More specifically, the invention relates to hydrogel particle-based workflows and compositions that normalize inhibitory effects of diverse sample matrices while enabling intact microbial capture and reproducible nucleic acid recovery for downstream molecular assays.

### BACKGROUND OF THE PRESENT INVENTION

Molecular extraction and purification technologies are foundational tools in modern biological, environmental, and clinical analysis. A wide variety of commercially available workflows have been developed to enable isolation of nucleic acids from diverse sample types, supporting downstream applications including polymerase chain reaction (PCR), reverse transcription PCR (RT-PCR), digital PCR, sequencing, and other molecular assays.

Existing extraction technologies typically rely on chemical lysis of target organisms followed by solid-phase nucleic acid purification using silica membranes, silica-coated magnetic beads, or analogous binding substrates. These approaches have been widely adopted due to their robustness, compatibility with automation, and ability to process many sample types with reasonable efficiency under controlled conditions.

For relatively homogeneous or low-inhibitor samples, such as buffered laboratory cultures or pre-treated clinical specimens, conventional extraction workflows can yield reproducible nucleic acid recovery suitable for sensitive molecular detection. Continued improvements in reagents, plastics, and automation platforms have further enhanced throughput and standardization within specific sample classes.

However, many biological and environmentally relevant samples exhibit substantial matrix-dependent variability. Complex matrices such as whole blood, urine, sputum, wastewater, food products, and environmental waters may contain endogenous substances including heme, salts, polysaccharides, proteins, lipids, humic substances, and other compounds that interfere with nucleic acid binding, washing, and enzymatic amplification.

In many conventional workflows, microbial targets are lysed directly within the native sample matrix. As a result, matrix-associated inhibitors are released concurrently with target nucleic acids and carried forward into downstream purification steps. Even when silica-based purification is employed, residual inhibitors may co-purify with nucleic acids, leading to reduced amplification efficiency, increased cycle threshold variability, or false-negative results.

To address these challenges, current extraction kits often require matrix-specific protocols, reagent formulations, and input volumes. While effective within narrowly defined contexts, such matrix-specific customization can increase operational complexity, reduce reproducibility across laboratories, and limit scalability when multiple sample types must be processed using a single analytical platform. In addition, workflows that rely on multiple centrifugation, filtration, or transfer steps may be labor-intensive, difficult to automate consistently, and prone to user-dependent variability, particularly when processing large numbers of samples or large input volumes.

Accordingly, there remains a need for improved sample preparation approaches that retain the advantages of existing nucleic acid extraction technologies while mitigating the effects of matrix-associated inhibitors and enabling standardized and simplified workflows. In particular, there is a need for methods that facilitate rapid and reproducible separation of target microbes or analytes from complex biological and environmental sample matrices, using approaches compatible with magnetic handling, automation, and scalable processing, prior to downstream molecular analysis.

Various approaches have been developed for the concentration and detection of microbial targets from biological and environmental samples. Known methods for concentrating microorganisms from water include membrane adsorption-elution techniques, tangential-flow and dead-end ultrafiltration, and chemical flocculation or precipitation, each of which may be effective for processing sample volumes ranging from milliliters to hundreds of liters under defined conditions.

Known documents describe certain magnetic particle-based systems for isolating biological analytes from complex samples. Such systems include engineered microbe-binding molecules and functionalized magnetic beads capable of capturing microbial targets from heterogeneous sample matrices. In many instances, these systems have been developed and optimized for specific sample types, including environmental surveillance matrices such as wastewater, which may contain suspended solids, organic contaminants, and diverse microbial communities, or clinical specimen matrices such as viral transport media (VTM) used for the collection and transport of swab-derived samples. However, such approaches are often configured for, and evaluated primarily within, the particular matrices for which they were developed, and may exhibit reduced performance, altered binding behavior, or limited applicability when applied to other sample types or matrices with substantially different physicochemical characteristics.

In many existing workflows, microbial targets are lysed directly within the native sample matrix to release nucleic acids for downstream purification. As a result, matrix-associated substances—including polysaccharides, humic acids, polyphenols, urea, proteins, salts, and lipids—may be released concurrently with target nucleic acids and co-purified during subsequent solid-phase extraction steps. Such co-purifying substances have been widely recognized to interfere with enzymatic amplification, leading to reduced sensitivity, increased variability, or false-negative results across a range of sample types.

Some approaches have addressed these challenges by developing matrix-specific protocols, sample-type-specific reagent formulations, or multi-step conditioning and secondary concentration procedures. While these approaches can be effective within narrowly defined contexts, they may increase operational complexity when multiple heterogeneous sample types must be processed using a single analytical platform. Other approaches have sought to reduce inhibitor carryover through dilution, dispersant treatment, or surfactant-mediated elution, but these techniques are generally applied at individual stages of existing workflows rather than as integrated components of a unified sample preparation method.

Accordingly, a need remains for methods and compositions that enable consistent processing of diverse biological and environmental samples using a simplified, standardized workflow. In particular, there is a need for approaches that facilitate the separation of intact microbial targets from complex sample matrices prior to lysis and nucleic acid recovery, while reducing the effects of matrix-associated inhibitors and maintaining compatibility with downstream molecular assays across heterogeneous sample types and volumes.

### SUMMARY OF THE INVENTION

The present invention provides methods, compositions, and kits for capturing and concentrating intact microbial targets from biological and environmental samples and preparing microbial nucleic acids for downstream analysis. The invention provides a unified workflow suitable for processing macro-volumes of complex fluids while reducing particle aggregation and maintaining compatibility with enzymatic amplification.

In one aspect, the invention provides a method for processing a sample comprising a microbial target, including viruses, bacteria, or fungi, using a matrix normalization workflow. The method comprises: (a) contacting the sample with a matrix conditioning composition; (b) capturing intact microbial targets using magnetically responsive hydrogel particles; (c) magnetically separating particle-bound microbial targets from the sample matrix to reduce soluble inhibitors; and (d) disrupting at least a portion of the captured microbial targets to release nucleic acids for subsequent purification and detection.

In another aspect, the invention provides magnetically responsive hydrogel particles comprising a polymeric hydrogel matrix that is intrinsically magnetic or magnetically addressable via association with a magnetic component. In certain embodiments, the hydrogel comprises one or more structural monomers and one or more affinity moieties selected to promote binding to microbial surface structures under defined conditions, thereby enabling rapid magnetic separation of captured microbial targets from bulk fluid.

In another aspect, the invention provides a matrix conditioning composition configured to normalize the sample matrix prior to microbial capture. In certain embodiments, the matrix conditioning composition comprises: (a) a buffering agent effective to condition the sample environment and promote reproducible capture; and (b) a surface-active agent configured to reduce unfavorable interfacial interactions, including particle aggregation and adhesion to biological materials or container surfaces. In certain embodiments, the matrix conditioning composition is added in an amount sufficient to deliberately dilute the sample matrix to reduce inhibitory substances while preserving capture of intact microbial targets.

In certain embodiments, the methods reduce or remove matrix-associated inhibitors, including polyphenols, polysaccharides, urea, humic substances, and proteins, thereby improving compatibility with PCR and RT-PCR across diverse sample types using a standardized workflow.

In certain embodiments, the methods enable concentration of microbial targets from sample volumes of about 0.1 mL to greater than 100 mL into elution volumes of about 10 μL to about 500 μL. In these embodiments, microbial targets remain intact during capture and washing, followed by lysis via thermal, chemical, mechanical, or enzymatic disruption to render nucleic acids accessible for purification and molecular analysis.

In certain embodiments, the methods are compatible with manual, semi-automated, and fully automated workflows and are applicable to biological, environmental, industrial, and food matrices, including whole blood, urine, serum, plasma, cerebrospinal fluid, saliva, bronchoalveolar lavage fluid, sputum, viral transport media, wastewater, surface water, potable water, culture media, and food matrices.

The following brief and detailed descriptions of the drawings are provided to explain possible embodiments of the present invention but are not provided to limit the scope of the present invention as expressed herein this summary section.

### BRIEF DESCRIPTION OF DRAWINGS OF THE PRESENT INVENTION

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 illustrates a representative workflow for processing a sample using the methods described herein. The workflow includes sample introduction, conditioning of the sample matrix, contact with magnetic hydrogel particles to capture microbial targets, magnetic separation of particle-bound targets from the sample matrix, lysis of captured targets to release nucleic acids, and downstream molecular analysis.

FIG. 2 exhibits a representative light microscopy image showing magnetic hydrogel particles associated with intact Escherichia coli cells prior to lysis. The image demonstrates physical binding of whole microbial cells to the particles following matrix conditioning and prior to downstream disruption.

FIG. 3 demonstrates a representative light microscopy image showing magnetic hydrogel particles bound to intact Escherichia coli cells. Individual bacterial cells are observed associated with the surface of the particles, indicating capture of intact microbes rather than released nucleic acids or cellular debris.

FIG. 4 is a representative image showing diverse sample matrices prior to processing (left) and visually clarified eluates obtained following matrix conditioning and magnetic hydrogel particle-based capture and separation (right), illustrating matrix normalization across heterogeneous samples.

FIG. 5 illustrates representative quantitative polymerase chain reaction (qPCR) cycle threshold (Ct) values obtained for a gram-positive bacterial target processed from multiple sample matrices using the methods described herein. The figure demonstrates detection of target nucleic acids across a range of biological and environmental sample types following magnetic particle-based capture and downstream molecular analysis, as described in Example 1.

FIG. 6 illustrates representative qPCR cycle threshold (Ct) values obtained for a gram-negative bacterial target processed from multiple sample matrices using the methods described herein. The figure demonstrates detection of target nucleic acids across a range of biological and environmental sample types following magnetic particle-based capture and downstream molecular analysis, as described in Example 1.

FIG. 7 illustrates representative qPCR cycle threshold (Ct) values obtained for a enveloped viral target processed from multiple sample matrices using the methods described herein. The figure demonstrates detection of target nucleic acids across a range of biological and environmental sample types following magnetic particle-based capture and downstream molecular analysis, as described in Example 1.

FIG. 8 illustrates representative qPCR cycle threshold (Ct) values obtained for a non-enveloped viral target processed from multiple sample matrices using the methods described herein. The figure demonstrates detection of target nucleic acids across a range of biological and environmental sample types following magnetic particle-based capture and downstream molecular analysis, as described in Example 1.

### DETAILED DESCRIPTION OF THE PRESENT INVENTION

The present specification discloses one or more embodiments that incorporate the features of the invention. The disclosed embodiment(s) merely exemplify the invention. The scope of the invention is not limited to the disclosed embodiment(s).

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to affect such feature, structure, or characteristic in connection with other embodiments whether or not explicitly described.

The methods and compositions described herein are suitable for molecular diagnostics and research applications, including epidemiology, wastewater surveillance, environmental monitoring, food safety testing, and biodefense, and support detection, identification, and nucleic acid isolation for amplification and sequencing.

As used herein, “sample” refers to a biological, environmental, or food matrix suspected of containing one or more microbial targets, including, without limitation, blood, plasma, serum, urine, saliva, cerebrospinal fluid, bronchoalveolar lavage fluid, sputum, viral transport medium, wastewater, surface water, groundwater, soil, sludge, industrial or agricultural water, and liquid or semi-liquid food matrices. “Microbial target” refers to viruses, bacteria, fungi, yeast, algae, protozoa, or parasites and may include intact organisms, partially intact organisms, fragments, or complexes. “Nucleic acid” refers to DNA or RNA, including single-stranded or double-stranded forms and fragmented or partially degraded forms. “Matrix normalization” refers to chemical and/or physical conditioning of a sample environment to reduce variability (e.g., pH, ionic strength, viscosity, detergents, lipids, fats, oils, organic content, and/or inhibitor burden) prior to capture of microbial targets.

In one aspect, the invention provides a matrix conditioning composition added to a crude or minimally processed sample prior to capture. In various embodiments, the conditioning composition reduces matrix-dependent variability, promotes consistent particle behavior, reduces aggregation or nonspecific adhesion, and improves reproducibility of microbial capture across diverse sample types and volumes.

In certain embodiments, the matrix conditioning composition comprises a buffering or conditioning agent effective to condition the chemical environment of the sample and promote reproducible capture. Suitable agents include, without limitation, amino acid-based buffers, organic acid buffers, inorganic buffering systems, or combinations thereof.

In certain embodiments, the matrix conditioning composition further comprises a surface-active agent that reduces unfavorable interfacial interactions in the sample, including particle aggregation and nonspecific adhesion to container surfaces. Suitable surface-active agents include, without limitation, non-ionic, zwitterionic, or amphipathic surfactants, including polysorbates and alcohol ethoxylates.

In certain embodiments, the conditioning composition is selected to provide a controlled sample environment (e.g., acidic, mildly acidic, or near-neutral) and/or is added in an amount sufficient to dilute the sample matrix, thereby lowering effective concentrations of soluble inhibitors while maintaining efficient capture and subsequent concentration of microbial targets.

In one aspect, the invention provides magnetically addressable hydrogel particles configured to capture microbial targets from conditioned samples and enable magnetic manipulation during capture, washing, and separation. In certain embodiments, the particles comprise a polymeric hydrogel matrix that incorporates a magnetic element (e.g., iron oxide, magnetite (Fe₃O₄), or another ferromagnetic or superparamagnetic material). In other embodiments, the hydrogel particles are non-magnetic and are configured to associate with a separate magnetic component to enable magnetic separation using an external magnetic field.

In certain embodiments, the hydrogel matrix comprises one or more structural monomers, optional crosslinkers, and one or more affinity moieties that confer charge-based, electrostatic, hydrophobic, and/or mixed-mode interactions with microbial surface structures. In certain embodiments, affinity moieties comprise anionic groups (e.g., carboxyl-containing monomers), cationic groups (e.g., amine-containing monomers), or combinations thereof, and capture performance is tunable by selection of affinity chemistry and conditioning parameters. In certain embodiments, the particles are configured as core-shell, composite, or modular structures integrating or coupling magnetic and capture functionalities.

In one aspect, the invention provides a method comprising: (a) contacting a sample with a matrix conditioning composition; (b) contacting the conditioned sample with magnetic hydrogel particles; (c) allowing microbial targets to associate with the particles; and (d) separating particle-bound microbial targets from the sample matrix using a magnetic field.

During magnetic separation, soluble inhibitors and non-bound matrix components remain in the supernatant and are removed, while microbial targets remain associated with the particles, thereby enabling inhibitor reduction prior to lysis.

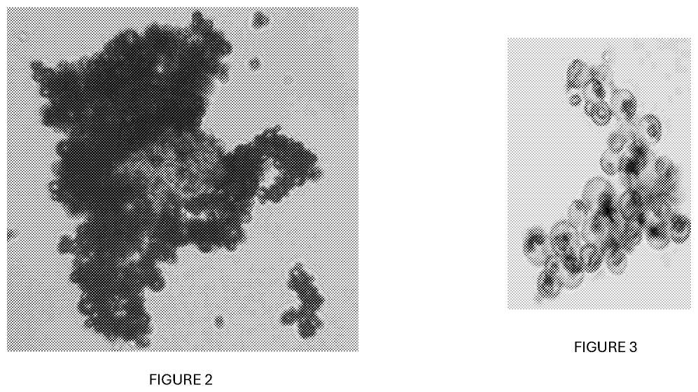

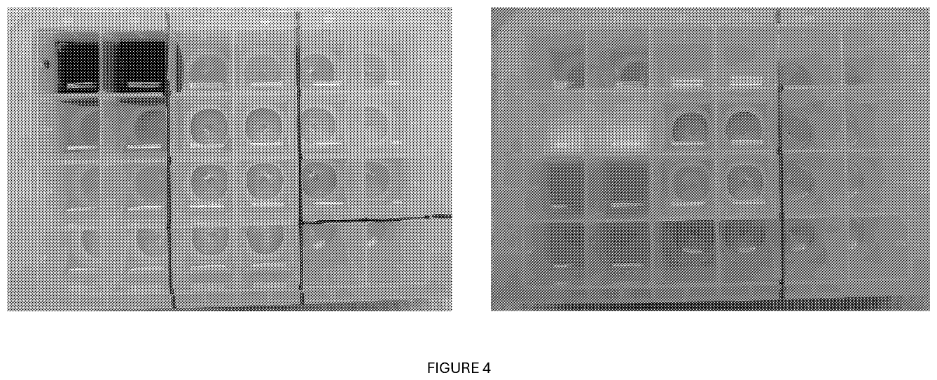

In certain embodiments, microbial capture occurs while microorganisms remain intact and unlysed. In such embodiments, microbial targets associate with the particles prior to any disruption step. As illustrated in FIGS. 2 and 3, microscopic analysis shows intact bacterial cells physically associated with the hydrogel particles, confirming that capture precedes disruption and nucleic acid liberation.

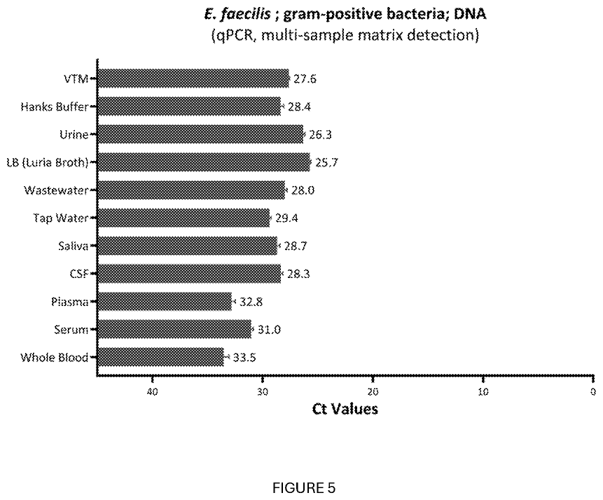

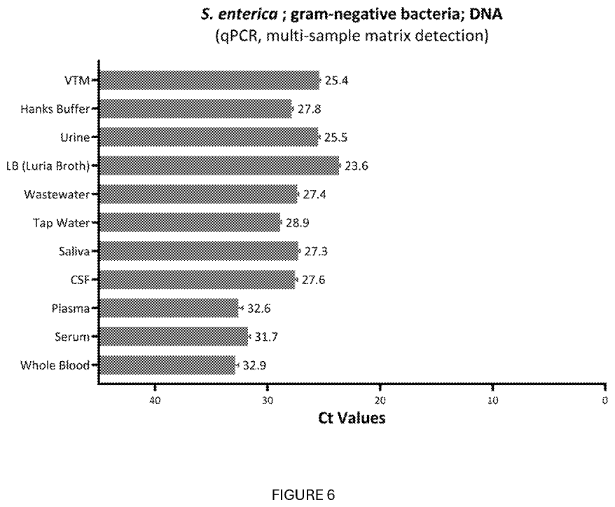

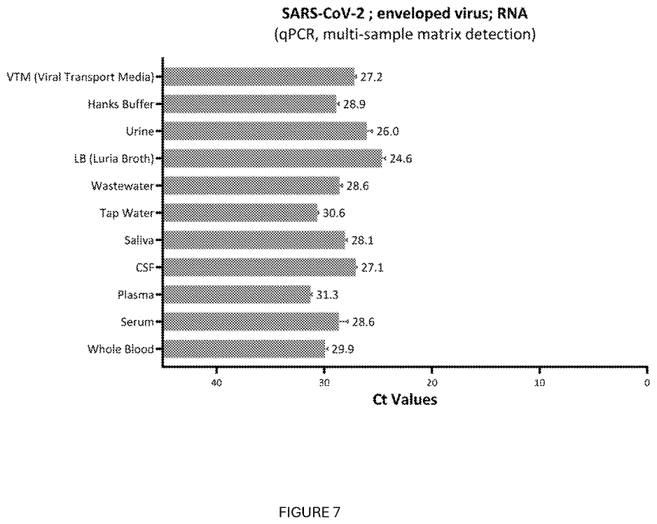

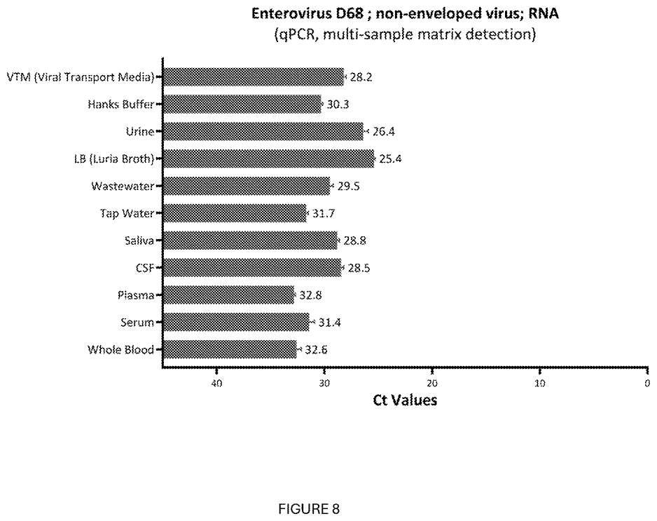

In certain embodiments, performance of the matrix normalization workflow is evaluated by quantitative PCR of nucleic acids recovered from microbial targets processed across multiple sample matrices. As illustrated in FIGS. 5-8, representative Ct values demonstrate consistent recovery following conditioning and particle-based capture across diverse sample types and processing conditions.

The workflow may be performed manually, semi-automatically, or using fully automated liquid-handling platforms and is scalable from microliter-scale inputs to volumes exceeding 100 mL.

Following capture and separation, microbial targets are disrupted to release nucleic acids. Disruption may be performed while targets remain associated with the particles or after transfer to a separate vessel.

Disruption methods include, without limitation, thermal, chemical, enzymatic, and/or mechanical lysis. Suitable lysis reagents include detergents, chaotropic agents, proteases, or combinations thereof.

In certain embodiments lysis reagents include agents capable of selectively releasing nucleic acids while leaving inhibitors and other non-nucleic acid material bound to the hydrogel matrix, further purifying the sample of interest.

In certain embodiments, released nucleic acids are purified using silica-based methods, including silica membranes or magnetic silica particles. Alternative purification approaches include precipitation-based or other solid-phase methods.

The purified nucleic acids are suitable for downstream enzymatic amplification and other molecular analyses.

Microbial targets processed using the methods and kits include, without limitation, bacteria (including Gram-positive and Gram-negative), fungi/yeast, viruses, and parasites. Exemplary bacteria include Escherichia coli, Vibrio cholerae, Pseudomonas aeruginosa, Neisseria meningitidis, Bacillus species, Staphylococcus aureus, and Clostridium species.

Viruses include, without limitation, coronaviruses, influenza viruses, retroviruses (including HIV), viral hemorrhagic fever viruses, encephalitic viruses, and reoviruses.

Nucleic acids isolated using the methods may be analyzed by PCR, RT-PCR, digital PCR, isothermal amplification, sequencing, and/or hybridization-based detection.

The invention further provides kits for isolating microbial targets and/or nucleic acids from a sample. In one embodiment, a kit comprises magnetic hydrogel particles, a matrix conditioning composition, and instructions for use.

In certain embodiments, the kit further comprises one or more lysis reagents, nucleic acid purification components, and/or reagents for molecular detection, including enzymes, oligonucleotides, or buffers.

In certain embodiments, the invention improves processing consistency across variable matrices by conditioning the sample environment prior to capture, thereby enabling a single standardized workflow for inhibitor reduction, intact microbial capture, and downstream nucleic acid analysis.

The invention will be further described with reference to the following examples; however, it is to be understood that the invention is not limited to such examples.

### EXPERIMENTAL EXAMPLES

The following examples are provided for purposes of illustration and description and are not intended to limit the scope of the present invention.

### Example 1: Manual Multi-Matrix Microbial Recovery Using Magnetic Separation

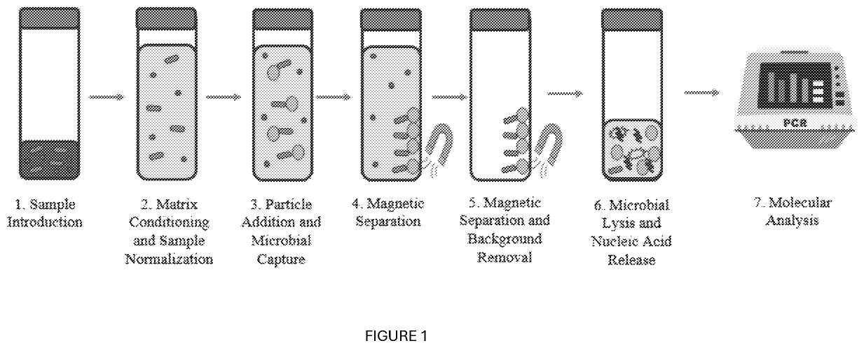

Eleven (11) distinct sample matrices were independently evaluated using a manual implementation of the methods of the present invention. Each sample matrix was separately prepared and processed to assess microbial recovery using a standardized workflow, as illustrated in FIG. 1.

The sample matrices included 1) whole blood, 2) urine, 3) serum, 4) plasma, 5) cerebrospinal fluid, 6) saliva, 7) viral transport medium, 8) LB broth, 9) wastewater, 10) tap water, 11) Hanks Buffer

In these examples, the microbial pathogens spiked into the sample matrices included, by way of illustration and not limitation, SARS-CoV-2, Enterovirus D68, Enterococcus faecalis, and Salmonella enterica. The selected pathogens represent enveloped and non-enveloped viruses as well as Gram-positive and Gram-negative bacteria.

For each matrix, a 1 mL aliquot of sample was transferred to a reaction vessel. Each sample was spiked with one or more of the microbial pathogens prior to processing. A volume of 4 mL of a proprietary buffer was added to each sample to condition the matrix. The samples were mixed to ensure uniform conditioning prior to microbial capture.

Magnetic hydrogel particles were added to each conditioned sample in an amount sufficient to capture microbial targets. The samples were incubated under conditions suitable to allow association of the microbial targets with the particles.

Following incubation, the magnetic hydrogel particles and associated microbial targets were separated from the sample matrix using a manual magnetic separator. The supernatant containing matrix-associated components was removed, and the particles were retained.

After capture, the captured microbial targets were lysed using a lysis procedure sufficient to release nucleic acids.

Released nucleic acids were purified using a silica-based purification method and eluted in a suitable buffer.

Purified nucleic acids were analyzed by quantitative polymerase chain reaction (qPCR) to assess microbial recovery.

Quantitative PCR analysis generated cycle threshold (Ct) values for each microbial target recovered from the respective sample matrices. The Ct values were used as a measure of detectable nucleic acid recovery following application of the disclosed matrix normalization and magnetic capture workflow.

As illustrated in FIG. 5-8, representative Ct values obtained across the tested biological and environmental sample matrices demonstrate consistent recovery of microbial nucleic acids following manual processing. The results indicate that the workflow supports detection of both viral and bacterial targets across matrices exhibiting substantial variability in composition and inhibitory content.

Collectively, the results of this example demonstrate that the disclosed methods enable reproducible recovery and detection of microbial nucleic acids from diverse sample types using a single, standardized manual workflow, supporting the utility of the matrix normalization approach for multi-matrix microbial analysis.

### Example 2: Automated Multi-Matrix Microbial Recovery Using Magnetic Probe-Based Processing

The sample matrices and microbial pathogens described in Example 1 were independently evaluated using an automated implementation of the methods of the present invention employing a magnetic probe-based processing system.

In these examples, the microbial pathogens spiked into the sample matrices included, by way of illustration and not limitation, SARS-CoV-2, Enterovirus D68, Enterococcus faecalis, and Salmonella enterica.

For each matrix, a 1 mL aliquot of sample was transferred to an automated processing vessel. Each sample was spiked with one or more microbial pathogens prior to processing. A volume of 4 mL of a proprietary buffer was added to each sample to condition the matrix.

Magnetic hydrogel particles were introduced into each conditioned sample. The automated system was configured to mix the samples and incubate them under conditions sufficient to allow microbial targets to associate with the particles.

Following incubation, the magnetic hydrogel particles were collected using magnetic probes that engage and transport the particles within the automated system. The probes were used to separate the particles and associated microbial targets from the sample matrix.

After capture, the captured microbial targets were lysed within the automated workflow using a lysis procedure sufficient to release nucleic acids. The magnetic probes were then used to transfer released nucleic acids or nucleic acid-binding particles to downstream purification steps.

Nucleic acids were purified using a silica-based purification method integrated into the automated workflow and eluted into a suitable elution buffer.

Purified nucleic acids were analyzed by quantitative polymerase chain reaction (qPCR). The automated magnetic probe-based workflow successfully recovered and enabled detection of microbial nucleic acids across all tested sample matrices, demonstrating compatibility of the disclosed methods with automated processing systems.

### Alternative Embodiments

Alternate embodiments of the present invention include variations in sample volume, order of operations, assay format, device configuration, and target organism. In certain embodiments, one or more steps of the disclosed workflow may be modified, omitted, combined, or reordered without departing from the scope of the invention.

In certain embodiments, the methods are applied to sample volumes ranging from microliter-scale inputs to large-volume samples exceeding 100 mL and enable concentration of microbial targets from dilute samples into reduced elution volumes suitable for downstream molecular analysis.

In certain embodiments, the magnetic hydrogel particles are introduced into the sample prior to, during, or after a conditioning step. In other embodiments, the particles are pre-loaded into a device, cartridge, membrane, or reaction chamber prior to sample introduction. Conditioning and capture steps may be performed sequentially or concurrently.

In some embodiments, captured microbial targets and/or released nucleic acids are analyzed using molecular assays including, without limitation, quantitative PCR, digital PCR, isothermal amplification, next-generation sequencing, nanopore sequencing, capillary electrophoresis, microfluidic analysis, and hybridization-based detection. In certain embodiments, nucleic acid quality, quantity, and/or integrity is assessed using spectrophotometric, fluorometric, and/or electrophoretic techniques, including automated fragment analysis.

In some embodiments, detection or analysis is integrated directly into the workflow or device. For example, an amplification cartridge, sequencing library preparation module, lateral flow format, or analytical sensor may be positioned downstream of capture and/or elution to enable direct delivery of analytes to the selected detection format.

In certain embodiments, the microbial targets include microorganisms selected from bacteria, viruses, fungi, yeast, algae, protozoa, and parasites. In some embodiments, bacterial targets include Gram-positive bacteria, Gram-negative bacteria, acid-fast bacteria, spore-forming bacteria, or combinations thereof. Exemplary bacterial targets include Escherichia coli, Salmonella species, Vibrio species, Pseudomonas species, Mycobacterium species, Staphylococcus species, Streptococcus species, Bacillus species, and Clostridium species.

In certain embodiments, viral targets include DNA viruses and RNA viruses, including single-stranded viruses, double-stranded viruses, positive-sense RNA viruses, negative-sense RNA viruses, segmented viruses, enveloped viruses, non-enveloped viruses, or combinations thereof. Exemplary viral targets include coronaviruses, influenza viruses, respiratory syncytial virus, adenoviruses, enteroviruses, noroviruses, herpesviruses, flaviviruses, filoviruses, and retroviruses.

In certain embodiments, fungal targets include yeasts and filamentous fungi. Exemplary fungal targets include Candida species, Aspergillus species, Cryptococcus species, and Histoplasma species.

In certain embodiments, protozoan or parasitic targets include Plasmodium species, Toxoplasma gondii, Giardia species, Cryptosporidium species, and Entamoeba species.

It will be understood that the foregoing lists are exemplary and non-limiting, and that the methods, compositions, and kits described herein are applicable to detection and analysis of additional microbial targets not expressly listed.

It should be understood that the sample processed using the methods of the present invention may comprise any biological, environmental, industrial, or food-derived matrix capable of being contacted with magnetic hydrogel particles.

In embodiments employing devices, cartridges, or fluidic systems, operating parameters such as flow rate, mixing, agitation, incubation time, and applied forces are selected to facilitate contact between the sample and the magnetic hydrogel particles and to enable magnetic separation and downstream processing.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

We claim:

1. A method for preparing microbial nucleic acids from a heterogeneous biological or environmental sample, comprising:
(a) contacting an unlysed sample comprising intact microbial targets with a matrix conditioning composition configured to condition physicochemical interactions within the sample;
(b) contacting the conditioned sample with magnetically responsive hydrogel particles configured to bind intact microbial targets;
(c) magnetically separating the magnetically responsive hydrogel particles and bound intact microbial targets from a liquid phase of the sample prior to microbial lysis;
(d) lysing the microbial targets after magnetic separation; and
(e) isolating microbial nucleic acids for molecular analysis.

2. The method of claim 1, wherein the matrix conditioning composition conditions one or more of pH, ionic strength, chelation state, surface wettability, dispersion behavior, or electrostatic interactions to promote reproducible microbial capture.

3. The method of claim 1, wherein the magnetically responsive hydrogel particles comprise a polymeric hydrogel matrix that is intrinsically magnetic or magnetically addressable through association with a separate magnetic component.

4. The method of claim 3, wherein the hydrogel matrix comprises one or more affinity monomers configured to interact with microbial surface structures through charge-based, electrostatic, hydrophobic, or mixed-mode interactions.

5. The method of claim 1, wherein magnetic separation is performed without centrifugation, filtration, or size-exclusion of intact microbes prior to capture.

6. The method of claim 1, wherein the matrix conditioning composition comprises a buffering or conditioning agent and a surface-active agent.

7. The method of claim 1, wherein the method enables processing of a plurality of distinct biological and environmental sample matrix types using a single standardized workflow.

8. The method of claim 1, wherein the sample comprises a biological fluid selected from whole blood, urine, plasma, serum, cerebrospinal fluid, saliva, bronchoalveolar lavage fluid, sputum, or viral transport medium.

9. The method of claim 1, wherein the sample comprises an environmental sample selected from wastewater, surface water, groundwater, industrial water, or combinations thereof.

10. The method of claim 1, wherein steps (a)-(e) are compatible with manual, semi-automated, or fully automated magnetic liquid-handling systems.

11. A kit for preparing microbial nucleic acids from heterogeneous sample matrices, comprising:
(a) magnetically responsive hydrogel particles configured to capture intact microbial targets; and
(b) a matrix conditioning composition configured to condition physicochemical interactions within a sample prior to microbial lysis.
(c) a lysis reagent where nucleic acids are isolated from the microbes and other inhibitory matrix elements present in the sample.

12. The kit of claim 11, further comprising instructions for performing a standardized workflow across a plurality of different biological and environmental sample matrices.

13. A system for matrix normalization and microbial nucleic acid preparation, comprising: magnetically responsive hydrogel particles; a matrix conditioning composition; and instructions directing a user to magnetically separate intact microbial targets from a sample matrix prior to lysis.
